(19) United States
(12) Patent Application Publication
(10) Pub. No.: US 2013/0025456 A1
(43) Pub. Date: Jan. 31, 2013

(54) AIR CLEANING FILTER MEDIA

(71) Applicant: Rick L. Chapman, Ventura, CA (US)

(72) Inventor: Rick L. Chapman, Ventura, CA (US)

(21) Appl. No.: 13/557,423

(22) Filed: Jul. 25, 2012

Related U.S. Application Data
(60) Provisional application No. 61/511,177, filed on Jul. 25, 2011.

(51) Int. Cl. B01D 46/00; A61L 9/00

(52) U.S. Cl. 95/285; 55/385.1

(57) ABSTRACT

An air purifying filter having vitamins, enzymes and/or other beneficial particles trapped between two light scrims of a carrier. The filter controls the release of the beneficial particles by using a predetermined size and shape of the openings in the air purifying filter media, and by reacting the particles with the air or other matter that passes through the filter, thereby causing the particles to shrink. The filter is also capable of retaining microorganisms by killing or otherwise controlling them due to the beneficial particles of the filter.

### CROSS-REFERENCES TO RELATED APPLICATIONS

This application claims the benefit of U.S. Provisional Application No. 61/511,177 filed Jul. 25, 2011. The prior application is hereby incorporated by reference.

### STATEMENT REGARDING FEDERALLY-SPONSORED RESEARCH AND DEVELOPMENT

(Not Applicable)

### REFERENCE TO AN APPENDIX

(Not Applicable)

### BACKGROUND OF THE INVENTION

This invention relates to an air purifying filter media having a vitamin, multiple vitamins or a combination of vitamins and/or enzymes or other antimicrobial material particles in a carrier web for gradual release into the atmosphere.

Air purifiers (also known as “air cleaners”) are machines that depend primarily on air purifying filters (also known as “air filters”) or electrostatic precipitation for removing unwanted materials, such as gaseous contaminants or suspended fine particles (e.g. dust), from the air. The process by which air filters remove particles from the air is well known, and typically includes the trapping of particles in the pores extending through a fibrous filter that are smaller than the particles themselves, but which allow air to flow therethrough.

Mechanical air filters typically filter air with synthetic, glass and/or cotton fibers woven or otherwise grouped in a web to remove dirt and/or dust particles, microorganisms and other particles from the air forced therethrough by a fan. Some filtration media also or alternatively use passive or active electrostatic attraction to retain particulate. The efficiency of particle capture is determined, at least in part, by the different types of filtration used.

Air purifying filters have been developed to suit specific circumstances and are available in various configurations, determined by the material to be removed from the air and its particle size. Filters are available in various shapes, such as flat, pleated, mini-pleated and separator style.

Conventional air purifying filters are unable to achieve complete removal of airborne microorganisms such as molds, bacteria and fungi for various reasons known in the field. Further, the microorganisms captured on the filters are difficult to control or kill, and the microorganisms may grow on the filters to later scatter about when air is forced through the filter, thereby causing secondary contamination. Existing air purifying filters have not given satisfactory results in air purification.

Japanese Patent Public Disclosure No. 246157/1994 discloses a cell adsorber which has the denatured product of a protein such as lysozyme, avidin or trypsin immobilized on a water-insoluble carrier. According to the disclosure, cells can be effectively separated or removed from a cell-containing solution by using the cell adsorber.

Japanese Patent Public Disclosure No. 236479/1995 discloses the lysozyme binding of an antimicrobial compound selected from among plant-derived antimicrobial compounds (e.g. perillaldehyde, cinnamaldehyde, salicylaldehyde, anisaldehyde, benzaldehyde and vanillin), antibiotics and synthetic antimicrobial agents, among others. The disclosure teaches that lysozyme bound to these antimicrobial compounds is useful in medicines, quasi-drugs and foods, among others.

Various inorganic antimicrobial materials have been proposed having antimicrobial metals such as silver, zinc and copper supported on inorganic carriers. Examples include (a) inorganic antimicrobial agents having silver, zinc and other ions supported on zeolite through ion-exchange, (b) inorganic antimicrobial agents having metallic silver supported on calcium phosphate through adsorption, (c) inorganic antimicrobial agents having silver ions supported on zirconium phosphate through ion-exchange, and (d) inorganic antimicrobial agents having silver complex salts supported on amorphous silicon oxide through occlusion. These inorganic antimicrobial materials are applied to fibers, plastics, films, paints and various other products, such as zeolites.

Various carriers have also been proposed for use in immobilizing the aforementioned enzymes and they include Japanese Patent Public Disclosure No. 91117/1994. This document discloses an air purifier having both a slime bacterial filter and a filter based on an antimicrobial polymer. The disclosure also teaches the typical use of polyurethane forms, polyethylene, polystyrene, polyacrylamides and/or various photocrosslinkable or photocurable synthetic polymers as the base for immobilizing slime bacteria produced lysing enzymes and antibiotics.

No conventional air filter produces air cleaning results that are satisfactory. Therefore, there is a need for an improved filter for improving the quality of air in a home or other building.

### BRIEF SUMMARY OF THE INVENTION

The invention contemplates an air purifying filter having particles containing one vitamin, multiple vitamins or a combination of one or more vitamins and/or enzymes on a surface of a filter media or support web composed of fibers. The fibers are preferably synthetic and are water-repellent. The air-purifying conditioning filter that can remove unwanted particles is combined with a layer of one or more vitamins, antioxidants, immune system support and/or enzymes that can saturate the atmosphere to have beneficial effect. These particles are released from the filter over time, thereby improving air quality, personal health and perception of cleanliness. By disposing antioxidants, immune system support and/or enzyme particles on the surfaces of carriers made of specified materials of specified sizes, the air-purifying filter controls distribution of the particles and removes microorganisms for a prolonged period of time.

The invention provides an air-purifying filter having particles made of one vitamin, multiple vitamins, antioxidants, immune system support and/or enzymes immobilized on the surface of a carrier, or between carrier layers of a filtration media. Antimicrobial purification of the air is thus aided by the dispersion of these preferably all-natural vitamins, antioxidants, immune system support and/or enzymes into the air when air is forced through the filter.

The invention further contemplates an air purifying filter having particles of one or more vitamins, antioxidants, immune system support materials and/or enzymes immobilized on the filter, wherein the filter is made of water-repellent synthetic fibers. One embodiment includes the use of vitamin C due to its well-known benefit to people who ingest it, and to its reduction of ozone in the air and absorption of materials that can be harmful or undesirable. One contemplated size of the vitamin C particles is in the range of 40 to 60 mesh, and preferably with about equal parts of each size. Other particles contemplated are desiccants that absorb moisture when the relative humidity of the air passing through the filter is high, and release moisture when the relative humidity is low, thereby reducing the high humidity that is common when there is cooking in a home, and increasing the humidity when it is sharply lower. Any antioxidants are also candidates due to their effect on the surrounding air and any “free radicals” therein.

As air passes through the air filtration media, suspended particulate, such as dust, and water molecules can be forced through the main layer of the purifying filter media, thereby striking the many different sizes of particles of the vitamins or other materials placed on or between the support layers. Most of these particles are water-soluble and will shrink over time, thus allowing them to pass through the support layer and enter the airstream over the lifetime of the completed filter. The particles are selected to have a range of sizes so that they are released at a relatively constant rate over the life of the filter as each particle shrinks to a size that can exit through a pore that is slightly larger than the particle.

### BRIEF DESCRIPTION OF THE SEVERAL VIEWS OF THE DRAWINGS

FIG. 1 is a schematic illustration showing the preferred filtration media and a pleat in media being formed.

FIG. 2 is a schematic illustration showing an alternative filtration media and a pleat in media being formed.

FIG. 3 is a schematic view in section illustrating an antimicrobial layer on a carrier layer.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose. For example, the word connected or terms similar thereto are often used. They are not limited to direct connection, but include connection through other elements where such connection is recognized as being equivalent by those skilled in the art.

### DETAILED DESCRIPTION OF THE INVENTION

U.S. Provisional Application Ser. No. 61/511,177 is incorporated in this application by reference.

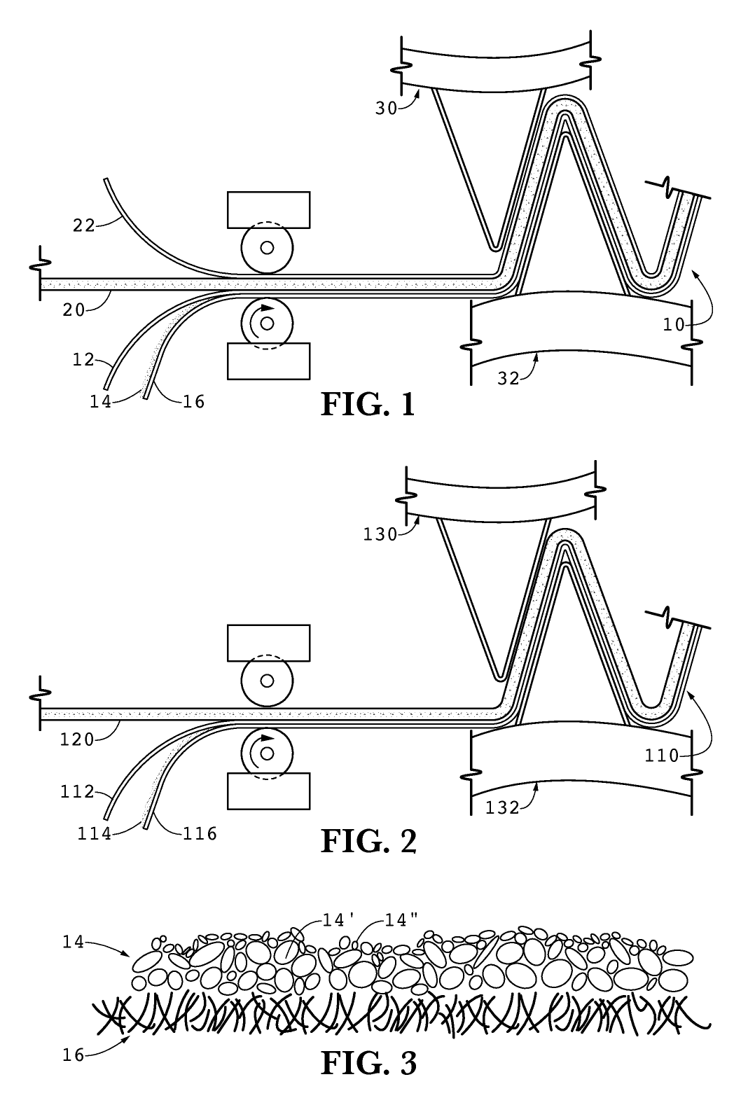

The preferred filter media 10 is shown in FIG. 1 during formation, where a downstream carrier layer 12 and an upstream carrier layer 22 are attached to opposite sides of, and sandwich therebetween, a filtration layer 20. The filtration layer 20 is a conventional layer that can be, but is not required to be, electrostatically charged, such as triboelectrically or otherwise. The terms “downstream” and “upstream” are relative terms that designate the position of one layer relative to another by the desired direction of flow of the fluid, such as air, through the media when it is in an operable orientation. Thus, because the carrier layer 12 is downstream of the filtration layer 20, air molecules pass through the filtration layer 20 before they pass through the carrier layer 12.

The carrier layers 12 and 22 support the filtration layer 20 during use when fluid is forced through the media. The carrier layers 12 and 22 are deformable and can retain their formed shape, such as during pleating, due to their material characteristics. The carrier web layers 12 and 22 can be the same or dissimilar materials, but each preferably provides little resistance to the flow of fluid through the media 10 and has substantial rigidity. These carrier layers can be non-woven synthetic, and preferably thermoplastic, fibers that form a batt or web. Examples of carrier web materials contemplated for use include various organic fibers and inorganic fibers such as cellulose fibers and non-glass synthetic fibers. These fibers may be used in different applications depending upon the characteristics. Preferably, the fibers of the carrier and cover layers are non-woven webs formed in conventional methods. The carrier layers 12 and 22 form lightweight layers over the filtration layer 20 that holds the filtration layer 20 in whatever shape the media 10 is formed into, such as a pleated shape. The non-carrier, air-purifying filtration layer 20 is not limited to any particular shape or configuration. The filtration layer 20 can be formed in a felt-like configuration, as in the form of a needled, punched nonwoven fiber batt, or it can use any mechanical bond between fibers, such as saturate bonding and/or point bonding. A preferred example of the filtration media 20 is described in U.S. application Ser. No. 13/280,500 filed Oct. 25, 2011, which is incorporated herein by reference. The desired characteristics of the carrier and cover layers depends upon the sizes of the particles being retained, but it is contemplated that for many of the embodiments of the invention, a range of about 1.3 to about 60 denier would suffice.

The cover layer 16 is downstream of the carrier layer 12 and the filtration layer 20, and has specified opening size as described in more detail below. The cover layer 16 is a porous material, preferably a non-woven fiber mat with strategically-sized openings, and can be the same material as the carrier layers 12 and 22. Particles 14 of vitamins or other beneficial materials are disposed between the cover layer 16 and the carrier layer 12. The particles 14 can be any of the materials described herein as having antimicrobial or other beneficial effect, including without limitation one type of vitamin, multiple types of vitamins, enzymes, antioxidants, immune system support, scented materials or any other such material.

In a preferred embodiment, the particles 14 are equal parts of each of several slightly different sized particles. With this arrangement, some of the particles 14 are released from the media 10 at different times during use of the media 10 as the particles 14 shrink. Shrinkage can occur due to mechanical reduction in mass by dust or other unwanted particles striking the particles 14, by removal of water from the particles 14 due to evaporation during use of the filter media 10, by chemical reaction with the air or other fluid flowing through the filter media 10, or by any other means by which the particles 14 are reduced in size. The vitamin, antioxidants, immune system support and/or enzyme particles 14 are then added between the two downstream layers 12 and 16 of the media 10. The cover layer 16 is specifically selected to have pore sizes and other features (e.g., electrostatic charge, etc.) that cause the cover layer 16 to retain the particles 14 initially, but release the particles gradually over time as the particles 14 shrink to a smaller size.

As shown schematically in FIG. 1, the heads 30 and 32 of a conventional pleating device form the self-supported pleats or unsupported pleats to be used in a filter made from the filtration media 10. The additional layer 22 on the upstream side of the filtration media 10 retains the formed pleat permanently, such as by chemical or thermal bonding between fibers of the layer 22, high temperature deformation and then cooling of thermoplastic fibers in the layer 22, or any other conventional means of retaining the deformed shape. Any or all of the carrier layers 12 and 22 and the cover layer 16 can be electrostatically charged. However, in the preferred embodiment only the filtration layer 20 has an electrostatic charge.

The materials that serve as the carrier layers 12 and 22 and the cover layer 16 of the air-purifying filter media 10 made according to the invention are not limited in any way as long as the filtration layer 20 is capable of performing as an air-purifying filter. That is, the layers 12, 16 and 22 must not increase the pressure drop so much as to substantially inhibit filtration by the filter media 10, or otherwise negatively and substantially affect the filtration layer 20. In order to ensure that an ample quantity of the particles 14 is used, it is preferred in the carrier and cover layers to use synthetic fibers that are water-repellent. Another preferred material is a low melt material having functional groups, as well as synthetic fibers.

In the preferred embodiment, the particles 14 in the filter media 10 are mechanically held in place due to their size relative to the sizes of the pores in the cover layer 16, which is the most downstream layer of the media 10. The particles 14 cannot initially pass through the openings in the cover layer 16 when air is forced through the media 10 because the particles 14, or at least most of the particles, are larger than the openings in the cover layer 16. However, as the particles 14 are affected by the air flowing through the media 10, they will preferably shrink, and then are gradually released from the media 10 through pores in the cover layer 16. This results in a release of consistent amounts of particles 14 into the air passing through the filter media 10, and provides the beneficial effect from the particles 14 passing through the air surrounding people in the building the filter media 10 is used in. Thus, the antimicrobial, antioxidant, scenting or other effect of the particles 14 occurs throughout the entire facility in which the filter media 10 is used.

As shown schematically in FIG. 3, the porous cover layer 16 holds back at least two types and sizes of particles 14 that are disposed upstream of the cover layer 16 (and downstream of the carrier layer 12, which is not visible in FIG. 3). Larger particles 14′ are placed downstream of smaller particles 14″ and all particles 14 are disposed upstream of the cover layer 16, so that, depending on the characteristics of the particles 14, their positioning will allow controlled release of the particles 14 over time. As an example, the particles 14 may be reduced in size by their vapor pressure and/or water solubility, thereby causing a time-controlled release as the particles 14 shrink due to reacting with air, moisture, undesirable particles in the air and other materials.

The particles 14 used in the invention can be a single vitamin, multiple types of vitamins, one or more antioxidants, one or more immune system supports and/or one or more enzymes, each used alone or in combination with others. The particles 14 are not limited in any particular way as long as they have a beneficial effect on the air quality in the facility in which they are used. It is contemplated that the particles will be vitamins, antioxidants, immune system support and/or enzymes, but other particles are contemplated that have a beneficial effect if released in a controlled manner. Examples of the mixtures or chemical compounds of such particles include, without limitation, vitamin C, vitamin D, vitamin E, vitamin A, quercetin, marshmallow extracts, licorice extract, pomegranate extract, astragalus, lytic enzymes, grape seed extract, Epigallocatechin gallate (EGCG, also known as epigallocatechin 3-gallate), thymus, taurine, L-tyrosine, l-glutamine, L-carnitine, equivalents and combinations of these, and any other particulate that has health-improving effect when used in the present invention. Any useful quantity of these particles is contemplated, and for vitamins a quantity of about 14 grams per square meter (g/m²) has been found to be useful. Of course, a range of 13 to 15 g/m² is also contemplated, as is a range of 10 to 20 g/m².

In FIG. 2 an alternative filtration media 110 is shown schematically by mounting the carrier layer 112 to the downstream side of the filtration layer 120, which media 110 is then pleated by heads 130 and 132 of a conventional pleating device that forms the self-supported pleats or unsupported pleats to be used in a filter made from the filtration media 110. The cover layer 116 is mounted to the downstream side of the carrier layer 112, and particles 114 including vitamins, antioxidants, immune system support and/or enzymes are disposed between the two layers 112 and 116. The cover layer 116 has specific size openings and the preferably different sized particles 114 seat against that layer so that air forced through the media 110 can force the particles 114 out of the pores in the cover layer 116 as the particles are able to do so. The lighter weight cover layer 112 may be pleatable and holds the particles 114 in place. There is the option of a pleated media using an electrostatic or a non-electrostatic filtration layer. It should be noted that there is no upstream carrier layer in the FIG. 2 embodiment.

This present invention will now be described in greater detail with reference to examples, comparative examples and tests. The invention is not limited in any way by these examples.

### EXAMPLE 1

The most downstream surface of a downstream carrier layer on media shown in FIG. 1 is a point bonded mixture of low melt fibers or sheath core fibers with very large fibers mixed with smaller denier fibers creating a sieving carrier screen. The preferred range of fibers is from about 1.3 to about 60 denier. This sieving carrier screen is designed with openings depending on the size of the particles held between the sieving carrier screen and the upstream layers. The openings are about 50 microns and the largest particles are about 70 microns, and therefore the particles smaller than 50 microns are slowly pushed by air around the other particles and through the openings to enter the airstream upon initial use. At the same time, the moisture-soluble particles (or the otherwise time-released particles) shrink and start to enter the airstream.

### EXAMPLE 2

This example uses the same downstream cover layer as the above Example 1 and has cover layer openings determined by the sizes of the particles. The media holds a single particle type and has a carrier layer upstream of the cover layer that is held to the cover layer by conventional point bonding. Upstream of the carrier layer is disposed a filtration media similar to the media disclosed in U.S. application Ser. No. 13/280,500 filed Oct. 25, 2011, which is incorporated by reference above. This filtration layer is an electrostatically charged non-woven fiber web that demonstrates very high efficiencies and very low flow resistance. On the upstream side of the filtration layer is another carrier layer that is very similar to the downstream carrier layer. The carrier layers are pleatable, permitting the media to be formed into a common pleated configuration of which commercial filters can be made.

### EXAMPLE 3

This example uses the FIG. 1 embodiment with a downstream cover layer having openings determined by the particle sizes. The filtration media is designed to hold a single vitamin, vitamins, antioxidants, immune system support and/or enzymes and has a carrier layer upstream of the cover layer holding the particles, and is bonded to the cover layer by point bonding low melt fibers or sheath core fibers with very large denier fibers mixed with smaller denier fibers. This creates a sieving screen at the most downstream point. As the moisture soluble particles shrink, the carrier web releases these particles and they enter the airstream. As this happens, the smaller particles of the other vitamin, vitamins, antioxidants, immune system support and/or enzymes slowly start entering the airstream.

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. A filter for removing particulate from a gas that flows through the filter, the filter comprising:
(a) a filtration media layer having a first major surface, a second major surface opposing the first major surface and a plurality of paths extending through the major surfaces of the filtration layer, at least some of the filtration media layer paths being smaller than at least some of the particulate;
(b) a cover layer having a first major surface and a second major surface, the second major surface mounted downstream of the filtration media layer and a plurality of paths extending through the major surfaces of the cover layer, at least some of the paths of the cover layer being smaller than a predetermined size; and
(c) antimicrobial particles on an upstream side of the cover layer, wherein at least most of the antimicrobial particles have a size that is greater than the predetermined size, and at least some of the particles are configured to shrink in size when exposed to the gas so that at least some of the particles that are greater than the predetermined size shrink to be smaller than the predetermined size and thereby are able to pass through the cover layer's paths.

2. The filter in accordance with claim 1, wherein the antimicrobial particles are vitamins.

3. The filter in accordance with claim 2, wherein the antimicrobial particles are a single type of vitamin.

4. The filter in accordance with claim 2, wherein the antimicrobial particles comprise at least two types of vitamins.

5. The filter in accordance with claim 1, wherein the antimicrobial particles are an enzyme.

6. The filter in accordance with claim 1, wherein the antimicrobial particles are an antioxidant.

7. The filter in accordance with claim 1, further comprising a carrier layer mounted to the second major surface of the filtration media layer and the second major surface of the cover layer.

8. The filter in accordance with claim 7, further comprising a carrier layer mounted to the first major surface of the filtration media layer.

9. The filter in accordance with claim 7, wherein the antimicrobial particles are selected from a group consisting of vitamins, antioxidants, immune system support and enzymes.

10. The filter in accordance with claim 7, wherein the antimicrobial particles are selected from a group consisting of vitamin C, vitamin D, vitamin E, vitamin A, quercetin, marshmallow extracts, licorice extract, pomegranate extract, astragalus, lytic enzymes, grape seed extract, Epigallocatechin gallate (EGCG), thymus, taurine, L-tyrosine, l-glutamine and L-carnitine.

11. The filter in accordance with claim 1, wherein the filter is pleated.

12. A method of using a filter for removing particulate from air that flows through the filter, the method comprising:
(a) installing a filter in a forced-air system, the filter including
(i) a filtration media layer having a first major surface, a second major surface opposing the first major surface and a plurality of paths extending through the major surfaces of the filtration layer, at least some of the filtration media layer paths being smaller than at least some of the particulate;
(ii) a cover layer having a first major surface and a second major surface, the second major surface mounted downstream of the filtration media layer and a plurality of paths extending through the major surfaces of the cover layer, at least some of the paths of the cover layer being smaller than a predetermined size; and
(iii) antimicrobial particles on an upstream side of the cover layer, wherein at least most of the antimicrobial particles have a size that is greater than the predetermined size;
(b) forcing air through the filter to cause at least some of the particles to shrink to a size that is smaller than the predetermined size, thereby allowing said shrunken particles to pass through the cover layer's paths and into the air surrounding the filter.
